(19) United States
(12) Patent Application Publication
(10) Pub. No.: US 2014/0336058 A1
(43) Pub. Date: Nov. 13, 2014

(54) METHOD AND KIT FOR CHARACTERIZING RNA IN A COMPOSITION

(71) Applicant: QIAGEN GMBH, Hilden (DE)

(72) Inventors: Erika Wedler, Hilden (DE); Holger Wedler, Hilden (DE)

(21) Appl. No.: 14/361,923

(22) PCT Filed: Nov. 30, 2012
(86) PCT No.: PCT/EP2012/074062

§ 371 Date: May 30, 2014

(30) Foreign Application Priority Data
Dec. 2, 2011 (EP) 11191749.8

(51) Int. Cl. C12Q 1/68

(52) U.S. Cl. CPC C12Q 1/6874; C12Q 1/6804
USPC 506/2; 435/6.11; 506/16

(57) ABSTRACT

The invention relates to a method for determining the sequence and/or quantity of a ribonucleic acid in a composition, comprising the steps of:
i. providing a composition comprising one or more ribonucleic acids molecules (RNA),
ii. hybridizing to said one or more RNAs, one or more two-part nucleic acid hybridization probes, wherein each probe comprises,
   a. a first nucleic acid molecule with a 3′-tail wherein said tail does not hybridize to an RNA in the composition,
   b. a second nucleic acid molecule with a 5′-tail wherein said tail does not hybridize to an RNA in the composition,
   c. wherein said first and said second nucleic acid molecules when and if hybridized to their target RNA lie on one single stranded RNA molecule separated from each other by between 2 and 1000 nucleotides,
iii. covalently linking the hybridized 5′-tail of said first nucleic acid molecule to the hybridized 3′-tail of said second nucleic acid, wherein the linking is done by means of reverse transcription and subsequent ligation,
iv. amplifying the linked molecules with primers that are specific for said first 3′-tail of said first nucleic acid molecule and said second 5′-tail of said second nucleic acid molecule and,
v. sequencing the amplification products by means of next generation sequencing.

### FIELD OF THE INVENTION

The present invention is in the field of molecular biology, diagnostics and more in particular expression profiling.

### BACKGROUND

Over the years, research in the field of trariscriptome analysis has progressed from candidate gene-based detection of RNAs using Northern blotting to high-throughput expression profiling driven by the advent of microarrays. Since 2006 next-generation sequencing technologies have revolutionized transcriptomics by providing opportunities for multidimensional examinations of cellular transcriptomes in which high-throughput expression data are obtained at a single-base resolution. Table 1 summarizes gene expression profiling milestones.

TABLE 1
Milestones for transcription analysis

| | | |
|---|---|---|
| 1995 | Serial Analysis of Gene | High throughput transcriptome-wide |
| | Expression (SAGE) | analysis of short sequences |
| | | (14 or 21 bp) derived from 3′ ends only |
| | | Labor and cost intensive cloning and |
| | | sequencing |
| | | High complexity of the process |
| 1995 | Gene expression | Low dynamic range (2-3 magnitudes) |
| | microarrays | Low specificity |
| | | Detection of noncoding RNAs, SNPs, |
| | | and alternative splicing |
| | | No detection of novel transcripts |
| | | Data noisy |
| 2006 | First transcriptome | Digital gene expression profiling (DGE) |
| | sequencing study using | Dynamic range: 5 magnitudes |
| | next generation | Single-base resolution |
| | sequencing (NGS) | Discovery of novel transcripts |
| | (454/Roche) | Detection of noncoding RNAs, SNPs, |
| | | and alternative splicing and transcript |
| | | aberrations |

Numerous protocols and commercial kits have been developed for mRNA-seq by next generation sequencing. Usually, the flow-chart of a standard transcriptome analysis includes 11 steps (The number and order of steps may slightly vary for several protocols and platforms. Library protocols for small RNA-seq based on RNA ligation follow a different workflow and are not considered here):
(1) Isolation of total RNA;
(2) Depletion of rRNA and/or enrichment of mRNA
(3) Fragmentation and size selection
(4) cDNA synthesis by reverse transcriptase reaction
(5) Second strand DNA synthesis
(6) End-repair
(7) Adapter ligation
(8) PCR enrichment of fragment library
(9) Cluster generation for subsequent sequencing (e.g. emPCR or bridge amplification)
(10) Sequencing and
(11) Data analysis

Enrichment of mRNAs and/or depletion of the rRNA are key steps for successful cDNA library generation and sequencing with minimal redundancy, because the major part of the total RNA consists of rRNA molecules.

For whole-transcriptome profiling of complex organisms like human many sequencing reads would have to be generated. Based on experimental data derived on Illumina sequencing platforms for the human transcriptome table 2 gives an overview of required number of reads and sequencing strategies for distinct analysis goals.

TABLE 2
Number of next-generation sequencing reads required for transcriptome
profiling for organisms with complex genomes like human

| Goal | Number Reads | Strategy |
|---|---|---|
| Array Quality | 2 * 10⁶ | 35 bp sequence length |
| Better than an array | 10⁷ | 35 bp sequence length derived |
| | | from polyA RNA |
| Quantitative cSNP | 50-100 * 10⁶ | 50-100 bp length from polyA |
| analysis for most | | RNA |
| transcripts incl. | | Paired-end sequencing |
| alternative splicing | | recommended |
| Complete annotation of | 500 * 10⁶ | 100 bp sequence length |
| an entire new | | Paired-end sequencing |
| transcriptome | | |

The enormous capacities for massive parallel sequencing on next generation sequencing platforms and the world-wide efforts in the genomics field have led to a tremendous improvement of our knowledge about the human genome and its expression profiles. Accordingly, we expect that almost all transcripts including splicing variants will be discovered in only just a few years.

However, complete analysis of complex transcriptomes is still expensive and labor intensive including the data analysis and extraction of biological and medical relevant information.

Furthermore, many working steps starting from RNA extraction to sequencing are time consuming, error-prone and make comparative studies difficult. Finally, many NGS machines do not have the capacity to generate enough reads for a whole complex transcriptome within one sequencing run.

to The present invention accomplishes the following improvements in this field, it leads to a significant improvement of sample preparation for RNA sequencing on NGS machines by reduction of the number of working steps, no no mRNA enrichment/purification no rRNA depletion are necessary, No adapter ligation is needed. The method now addresses NGS machines with limited sequencing capacity by targeted gene expression) profiling (gene panel oriented assays), the method enables multiplexed analysis by indexing, analysis of gene expression levels, analysis of known (examples 1 and 2) and unknown (example 3) splice site variants including as well as their quantification including single-base resolution and hence SNP detection (see example 3). The method provides for a large digital dynamic range.

Further, in contrast to U.S. Pat. No. 7,361,488, no support is needed and what is more important the sequence of the in vivo RNA is determined rather than the hybridized oligonucleotide detected. This difference is quite substantial.

### DEFINITIONS

A “composition” herein is an aqueous solution comprising at least one or more ribonucleic acid molecules.

A “first nucleic acid molecule with a 3′-tail wherein said tail does not hybridize to an RNA in the composition” is an oligonucleotide which has two parts, a first part is able to bind its RNA target (specifically) if the target is present in the composition and a second part which does not hind an RNA in the composition.

A “second nucleic acid molecule with a 5′-tail wherein said tail does not hybridize to an RNA in the composition” is an oligonucleotide which has two parts, a first part is able to bind its RNA target (specifically) if the target is present in the composition and a second part which does not bind an RNA in the composition.

### DETAILED DESCRIPTION OF THE INVENTION

The invention relates to a method for determining the sequence and/or quantity of a ribonucleic acid in a Method for determining the sequence and/or quantity of a ribonucleic acid in a composition, comprising the steps of:
(i) providing a composition comprising one or more ribonucleic; acids molecules (RNA),
(ii) hybridizing to said one or more RNAs one or more two-part nucleic acid hybridization probes, wherein each probe comprises,
   (a) a first nucleic acid molecule (DNA) with a 3′-tail wherein said tail does not hybridize to an RNA in the composition,
   (b) a second nucleic acid molecule (DNA) with a 5′-tail wherein said tail does not hybridize to an RNA in the composition,
   (c) wherein said first and said second nucleic acid molecules when and if hybridized to their target RNA lie on one single stranded RNA molecule separated from each other by between 2 and 1000 nucleotides,
(iii) covalently linking the hybridized first nucleic acid molecule to the hybridized second nucleic acid, wherein the linking is done by means of reverse transcription and subsequent ligation,
(iv) amplifying the linked molecules with primers that are specific for said first 3′-tail of said first nucleic acid molecule and said second 5′-tail of said second nucleic acid molecule,
(v) sequencing the amplification products, preferably by means of next generation sequencing, wherein prior to the amplification step (iv)
(vi) the hybrids of target RNA and linked molecules of step (iii) are isolated by capturing the hybrids with an antibody that is specific for a DNA/RNA hybrid.

The separation of the two nucleic acids molecules is ideally between 2 and 1000, 5 and 500 and most preferably between 35 and 150 nucleotides. The two molecules are deoxyribonucleic acids (DNA) or comprise DNA such that the antibody is functional and binds the hybrid,

U.S. Pat. No. 7,361,488 discloses a method wherein nucleic acid probes which have hybridized to an RNA target are ligated together and then subsequently amplified and detected. The drawback of this method is that the detection occurs by means of the probes which were originally added to the reaction. No de novo in vivo sequence is determined (only known sequences are detectable) and the detection is only indirect as one must assume, based on the detection of the probe that a certain RNA was present. New and unknown sequences to are not detectable. But, was that RNA present? That remains unclear when using the method of U.S. Pat. No. 7,361,488. The present invention solves this problem as the section steps allow for, for the first time the actual sequence determination of defined RNA stretches from, e.g. mRNA transcripts.

Probes and primers of the present invention are designed to have at least a portion be complementary to the polyadenylated mRNA target sequence or an RNA from another species, such that hybridization of the polyadenylated mRNA target sequence or the RNA from the other species and the probes of the present invention occurs. As outlined below, complementarity need not to be perfect; there may be any number of base per mismatches which will interview hybridization between the polyadenylated mRNA target sequence in a single stranded nucleic acid of the present invention. However, if the number of mutation is so great that no hybridization can occur under then the sequence is not a complementary polyadenylated mRNA target sequence (the same applies to an RNA from another species). Hence, the probes described in claim 1 must be “substantially complementary” which herein means that the probes are sufficiently complementary to the polyadenylated mRNA (or RNA from the other species) to hybridize under normal reaction conditions and preferably give the required specificity.

A variety of hybridization conditions may be used in the present invention including high, moderate and low hybridization conditions; see for example Maniatis et. al,, Molecularing Cloning: A Laboratory Manual, 2^{nd} Edition, 1989 and short protocols in Molecular Biology.

Stringent conditions are sequence-dependent and will be different in different circumstances. Longer sequences hybridize specifically at higher temperatures, Generally, stringent conditions are selected to be about 5 to 10° C. lower than the thermal melting point (Tm) for the specific sequence at a defined ionic strength. The TM is the temperature (under defined ionic strength, ph nucleic acid concentration) at which 50% of the probes complementary to the target hybridize to the polyadenylated mRNA target sequence at equilibrium (as the target sequences are present in excess at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7 to 8.3 and the temperature is at least about 30° C. for short probes (e.g. 10 to 50 nucleotides) arid at least about 60° C. for long probes (e.g. greater than 50 nucleotides). Stringent conditions may also be achieved preferentially herein with the udition of helix destabilizing agents.

The method as designated above will make use of a large number of two-part nucleic acid hybridization probes also termed herein as target probes that are used in particular in a multiplex fashion. A plurality of these probes is used and means 10 or more of such probes. Preferably between 15 and 100, more preferably between 100 and 500, even more preferably between 100 and 1000.

As outlined above, the first nucleic acid molecule has a 3′-tail and the second nucleic acid molecule has a 5′-tail. These are so called universal priming sites. By “universal priming site” herein is meant a sequence of the probe that will bind a PCR primer for amplification. Each probe preferably comprises an upstream universal priming site and a downstream universal priming site. Herein, these are located on said first nucleic acid molecule and said second nucleic acid molecule. Again, “upstream” and “downstream” are not meant to convey a particular 5′- or 3′-orientation and will depend on the orientation of the system, Preferably, only a single upstream universal priming site and a single downstream universal single priming site is used in a probe said, These sequences are generally chosen to be as unique as possible given the particular assays and host genomes to ensure specificity of the assay.

It is preferred that the isolation is done by capturing the hybrids with an antibody that is specific for a DNA/RNA hybrid and said antibody is bound to some sort of a solid phase, such as a magnetic particle.

Even better results are achieved if the RNA is enzymatically digested prior to the amplification step (iv).

The length of the first nucleic acid molecule with a 3′-tail wherein said tail does not hybridize to an RNA in the composition and the second nucleic acid molecule with a tail wherein said tail does not hybridize to an RNA in the composition is preferably between 5 and 100 nucleotides, preferably between 10 an 50 nucleotides and more preferably between 15 and 30 nucleotides.

The tail of these molecules, wherein said tail does not hybridize to an RNA in the composition is preferably between 5 and 100 nucleotides, preferably between 10 an 50 and more preferably between 15 and 30 nucleotides.

In one optional embodiment the first nucleic acid molecule and/or second nucleic acid molecule comprise a further barcode sequence, determined not to bind the target RNA. (see also FIG. 22).

The further barcode sequence is preferably from 5 to 6 nucleotides in length it may be between 3 and 20, 5 and 8 nucleotides in length. Other lengths may be envisioned.

These molecular barcodes are generated by introduction of random nucleotides between universal tail and target specific sequences of the probe. It allows differentiation between fragments derived from a target RNA molecule and copies generated during PCR amplification causing a sequence bias (FIG. 22). Such barcodes may be integrated in both probes A or B, respectively. For example: A barcode consisting of 5 random nucleotides allows differentiation of maximum 45=1024 copies of a RNA molecules, and two barcodes with 45 random nucleotides, each, allow differentiation of 45*45=1,048,576 molecules.

The first nucleic acid molecule with a 3′-tail wherein said tail does not hybridize to an RNA in the composition and the second nucleic acid molecule with a 5′-tail wherein said tail does not hybridize to an RNA in the composition, are specific for a nucleic acid sequence selected from the group of, a human nucleic acids sequence, a viral sequence, a bacterial sequence, an animal sequence and a plant sequence. Hence, the specific sequence may be specific for a human nucleic acids sequence, a viral sequence, a bacterial sequence, an animal sequence and a plant sequence.

Probes and primers of the present invention are designed to have at least a portion be complementary to the poly-A related mRNA target sequence or an RNA from another species, such that hybridization may occur.

Preferably the sequence for which they are specific is mRNA, it may be an exon-exon junctions and/or 5′ and 3′ UTR region.

Preferably, the next generation sequencing method applied is selected from the group of,
(i) Illumina: HiSeq 2000, HiSeq 1000, Genome Analyzer IIx, MiSeq, HiScanSQ (chemistry Sequencing by synthesis),
(ii) Roche: Roche 454 FLX, GS Junior (chemistry: Pyrosequencing),
(iii) Invitrogen: SOLiD 5500 Series, (chemistry: sequencing by oligo ligation),
(iv) Invitrogen: IonTorrent PGM (chemistry: semiconductor technology),
(v) Pacific Biosciences: PacBio RS system (chemistry: single molecule, real-time (SMRT™) sequencing). In other word the sequencing method may Sequencing by synthesis, pyrosequencing Sanger sequencing, sequencing by oligo ligation, semiconductor technology or single molecule real-time (SMRT™) sequencing. Preferably the read lengths of the next generation sequencing method used are as high as possible but that must not be necessary, They may be, e.g., single end 36 or up to 150 bases or 2×36 up to 2×150 bases paired end (Illumina), single end up to 50 bases or 75 bases paired-end: 75 bp ×35 by (SOLiD), up to 400-500 bases (Roche), or up to 100-200 bases single end or paired end (Ion Torrent)

Illumina single end reads up to 150 bases or paired end up to 300 bases (2×50 bases) are preferred.

Preferably in the next generation sequencing method 25 to 500 bases are read per read, preferably between 25 and 200 nucleotides and more preferably between 25 and 150 nucleotides are read per read. Alternatively to single reads, paired end readings may be applied.

The method does to a certain extent depend on the concentration of the first nucleic acid molecule with a 3′-tail wherein said tail does not hybridize to an RNA in the composition and the second nucleic acid molecule with a 5′-tail wherein said tail does not hybridize to an RNA in the composition which ideally is between 1 fM and 1000 nM.

The invention also relates to a kit comprising a first nucleic acid molecule with a wherein said tail does not hybridize to an RNA in the composition, a second nucleic, acid molecule with a 5′-tail wherein said tail does not hybridize to an RNA in the composition, wherein said first and said second nucleic acid molecules when and if hybridized to their target RNA lie on one single stranded RNA molecule separated from each other by between 2 and 1000 nucleotides and an antibody which is specific for an RNA/DNA duplex hybrid molecule.

The invention is best illustrated by Example 3 and FIG. 12. The other examples and figures serve for a better understanding of the present invention.

### FIGURE CAPTIONS

FIG. 1:

A standard RNA sample preparation workflow for whole transcriptome library generation and sequencing is shown. B. Simplified workflow for example 1 of the invention. Capture DNA probes consist of nucleotide sequences with homology to targeted mRNAs (light blue, dark blue and brown lanes) and 5′ (red) as well as 3′ (green) universal adapter tails without homology to the transcriptome. After hybridization RNA/DNA hybrids are captured with paramagnetic bead-coupled antibodies (blue Y). Finally, captured oligonucleotides libraries are amplified with universal primers to introduce sequence motifs for cluster generation, sequencing primer annealing and optionally for indexing allowing sample multiplexing.

FIG. 2:

The experimental workflow for example 1—experiment 1 is shown. Varying probe amounts in a range of 2 magnitudes were hybridized with constant amount of total RNA. Results of the capturing experiment were analyzed in 2 independent experiments by quantification of both captured probes and mRNA (target and off-target).

FIG. 3:

An amplification plot (left) and melting curves (right) of captured hybridization probes derived from 2 different experiments (upper lane: 1st experiment, lower lane: repeated 2nd experiment) is shown. Similar amplification curves and ct values indicate similar amounts of captured oligo probes. The melting curves are similar to the oligo mixture before hybridization. This indicates that the amplification plots obtained after SybrGreen qPCR are specific for the probes used.

FIG. 4:

SybrGreen amplification plots of 2 independent experiments (upper row: 1st experiment; lower row: 2nd experiment) are shown. Left: Amplification plots of the ACT cDNA (target region). Right: Amplification plots for RPT13a cDNA (off-target region). Control reactions indicated by K were performed after cDNA synthesis using 200 ng total RNA without hybridization. Template amounts for control reactions are not comparable with amounts obtained after hybridization. Therefore, ct values are not comparable as well. Amplification plots and ct values of the cDNAs derived from captured ACT mRNA are very similar independent from the amount of probes used for hybridization. Amplification plots and ct values for the cDNA derived for the off-target region (RPL13a mRNA) are identical with the negative control (hybridization without probes), indicating successful enrichment for the targeted RNAs. The reason for amplification curves of the negative control might be found in the nature of SybrGreen PCR and/or some extent of unspecific capturing without probes by the beads.

FIG. 5:

A column chart of the VCR data after hybridization and reverse transcriptase (RT) reaction for target and off-target mRNAs is shown.

FIG. 6:

An experimental workflow for example 1—experiment 2 is shown. Varying amounts of total RNA in a range between 50 ng and 1000 ng were hybridized with 0.7 nmol capture probe mixture (5.5 pmol each). Captured probes were analyzed exactly as in experiment 1 by SybrGreen qPCR or reverse transcriptase reaction and SybrGreen qPCR, respectively.

FIG. 7:

SybrGreen qPCR results of 2 independent experiments for quantification of captured probe oligos are shown. qPCR was performed using primers, which are homolog to the tailed sequences of the probes. In addition to the determination of ct values dissociation curves were generated to show the specificity of PCR products (for details see protocol in the appendix RSE0205). Unfortunately, PCR experiments without template resulted partially in amplification products. However, the ct values obtained for these controls were significant higher compared to those obtained for samples with template and therefore we do not expect a significant influence on the results.

FIG. 8:

FIG. 8 shows the RT-qPCR results of RNAs picked. mRNA of the ACT gene was picked for detection of the targeted RNA and compared with the mRNA of the RPL13a gene as off-target. Whereas the yield of captured RPL13a mRNAs remains nearly even, the captured mRNA yields of the ACT gene correlate with the total RNA amounts used for hybridization.

SybrGreen qPCR results after reverse transcription of captured RNA using random 9mer primers are shown. With increasing RNA amounts the yields of captured RNAs increased, as indicated by decreased ct values. Whereas total RNA amounts used for hybridization and captured targeted RNAs (detection of mRNA from the ACT gene) show strong correlation, the yield of the off-target mRNA from RPL13a gene remains nearly constant, Hybridization with total RNA amounts doubled results in a delta-ct value of approximately −1 for targeted RNAs. Ct values obtained for samples without hybridization can not be compared with data obtained from capture experiments because of different RNA amounts used.

FIG. 9:

A general workflow for example 2 of the invention is shown. Hybridization and ligation mediated gene expression profiling. Long blue, black, brown and pink lanes indicate targeted mRNAs. Short lanes and arrows in the same colors indicate reverse complementary oligo probes to their target. Phosphorylation of the 5′ end of oligoA is not shown. Short red and green lanes and arrows indicate universal 5′ and 3′ tails of the probes, respectively. Primers for enrichment PCR are shown in red and yellow or green and light blue, respectively to indicate homologies to the probe tails as well as sequencing specific ends.

FIG. 10:

An experimental design for ligation of oligonucleotides after hybridization on RNA templates including expectation for the results is shown. Identical colors of arrows and lanes indicate primers and probes with homolog sequences. Phosphorylation of the 5′ end of probes DDX56A and DDX67A is not shown.

FIG. 11:

Ct values of hybridized and ligated oligo probe DDX67A+B on RNA templates DDX56 and DDX67 after SybrGreen PCR in different buffer systems are shown.

FIG. 12:

A schematic presentation of the workflow for example 3 of the invention is shown.

FIG. 13:

A workflow for example 3—experiment 4 of the invention is shown.

FIG. 14:

Agilent 2100 analysis of generated probes after PCR enrichment (endpoint PCR) is shown. Fragments, indicated with green tagged PCR primer pairs were expected and fragments, indicated with red tagged primer pairs were expected to fail analysis. Only expected fragments were detected in correct size.

FIG. 15:

FIGS. 15 to 20 show sequence chromatograms of successful fused probe oligos. All PCR fragments were sequenced on both strands using PCR amplification primers. In all cases the expected sequences were found, indicating the accuracy of the RT polymerase and ligase reaction.
Hybridization: A1
Template: RNA DDX56
Probe: DDX56E+F
Sequencing Primer: M13f

FIG. 16:
Hybridization: A2
Template: RNA DDX67
Probe: DDX67E+F
Sequencing Primer: pUCF

FIG. 17:
Hybridization; B1
Template: RNA DDX56
Probe: DDX56C+D
Sequencing Primer: pUCF

FIG. 18:
Hybridization; B2
Template: RNA DDX67
Probe: ⁻DDX67G+14
Sequencing Primer: M13f

FIG. 19:
Hybridization: C1
Template: RNA DDX56
Probe: DDX56E+F
Sequencing Primer: M13f

FIG. 20:
Hybridization: C2
Template: RNA DDX67
Probe: DDX67J+K
Sequencing Primer: pUCF

FIG. 21:

Schematic presentation of the workflow of the invention (Example 3), 1, Total RNA. II. Hybridization of total RNA with a mixture of target specific DNA probes. Tailed probes A and B match in a distance to their target. III. Closing gap between oligonucleotide A and B by RT polymerase reaction and ligation in presence of ATP. Phosphorylation of the 5′ end of probe B is not shown. IV. Enrichment of newly synthesized cDNA molecules by antibody based purification of the DNA/RNA hybrids. V. Release of newly synthesized DNA by denaturation and RNAse treatment. VI. Enrichment of targeted cDNAs by PCR before sequencing.

FIG. 22:
Hybridization of RNA with molecular barcoded probes,

### EXAMPLES

### Example 1

mRNA Profiling by Hybrid Capture Technology:

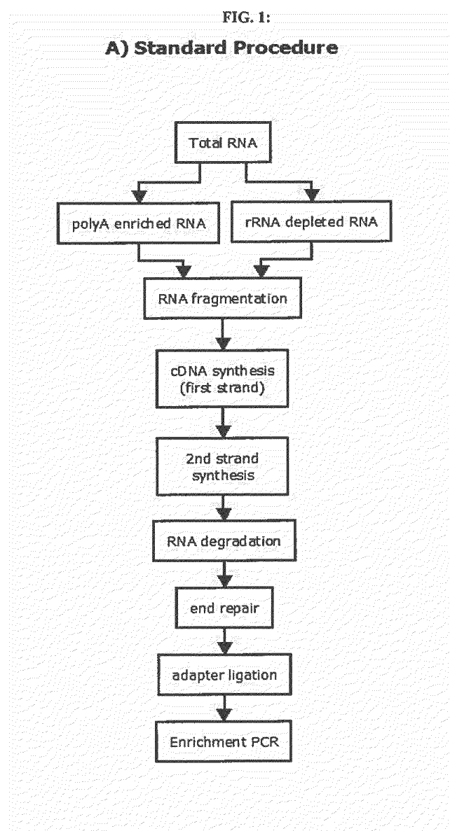

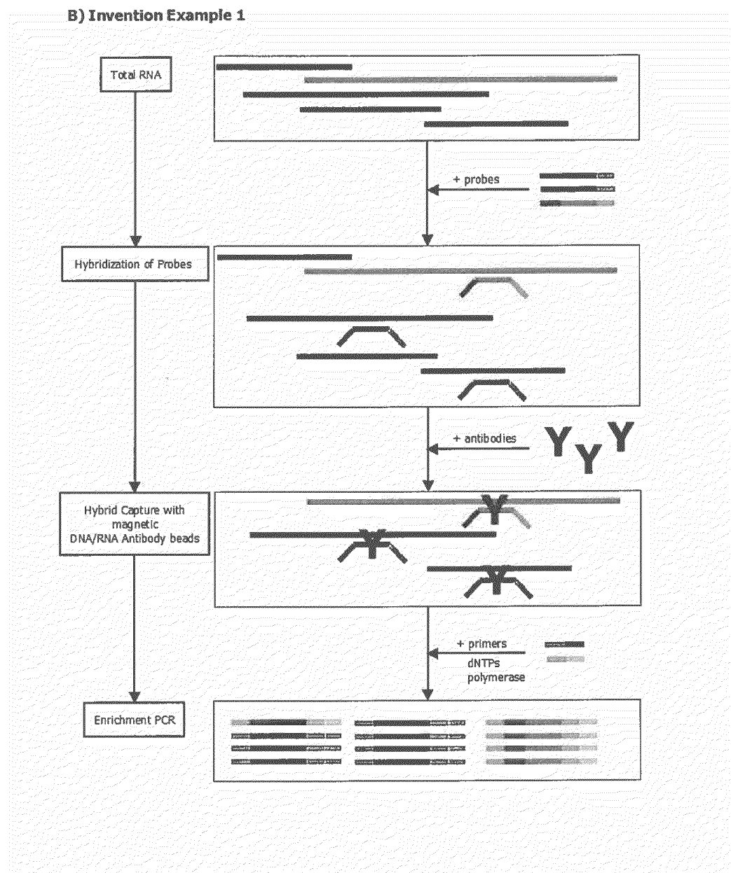

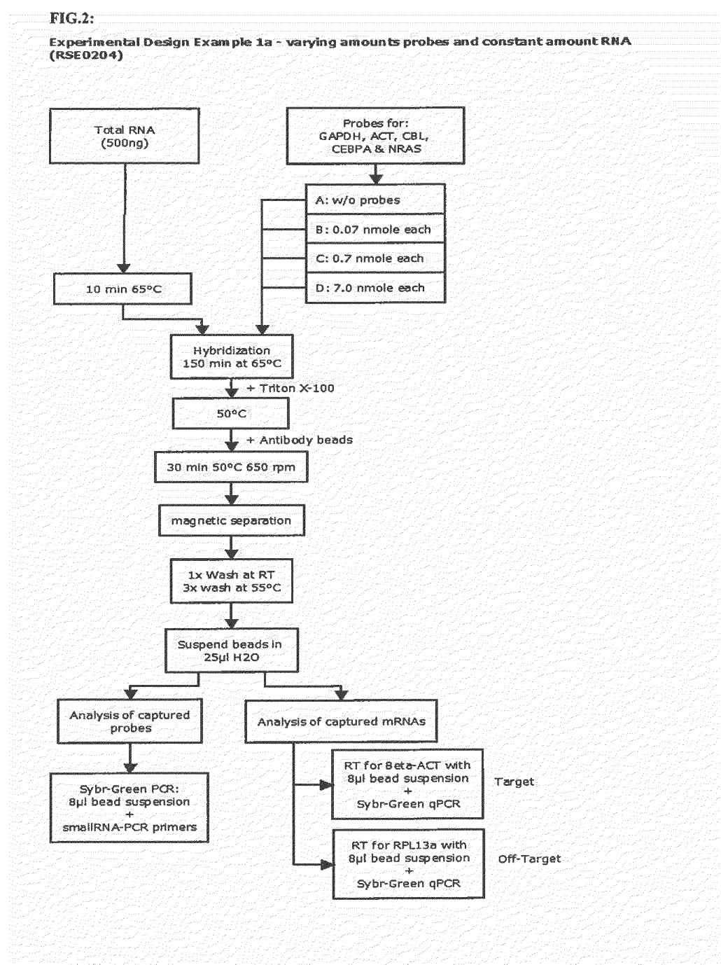

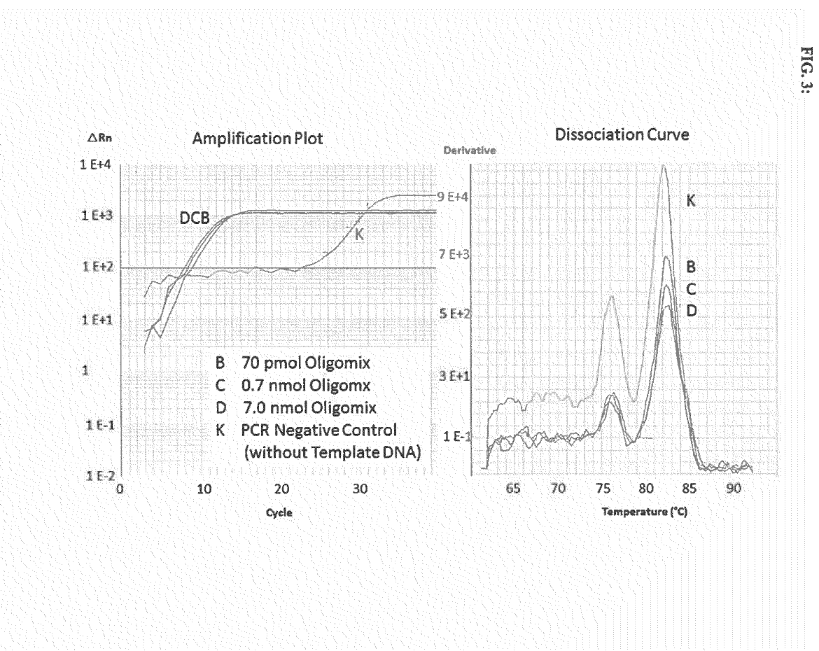

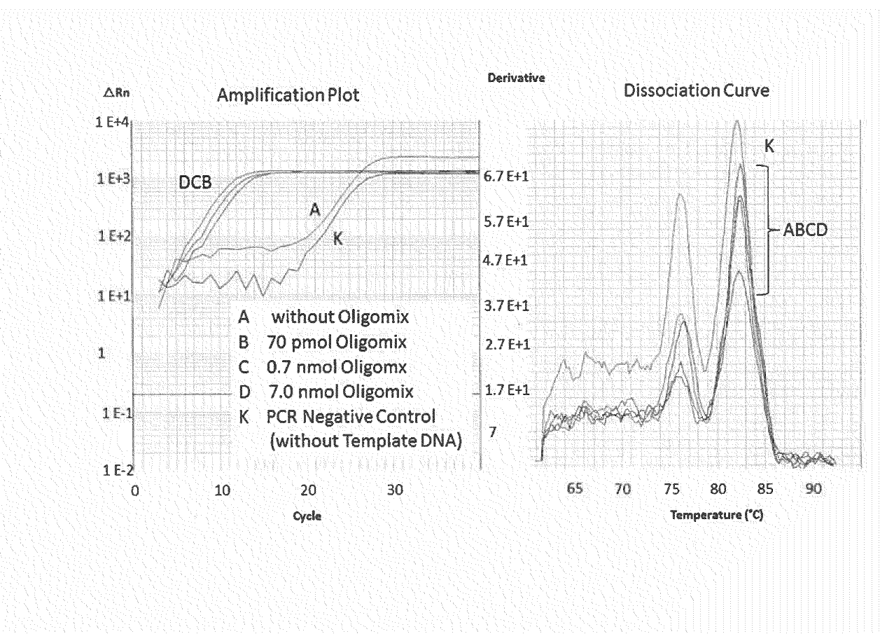

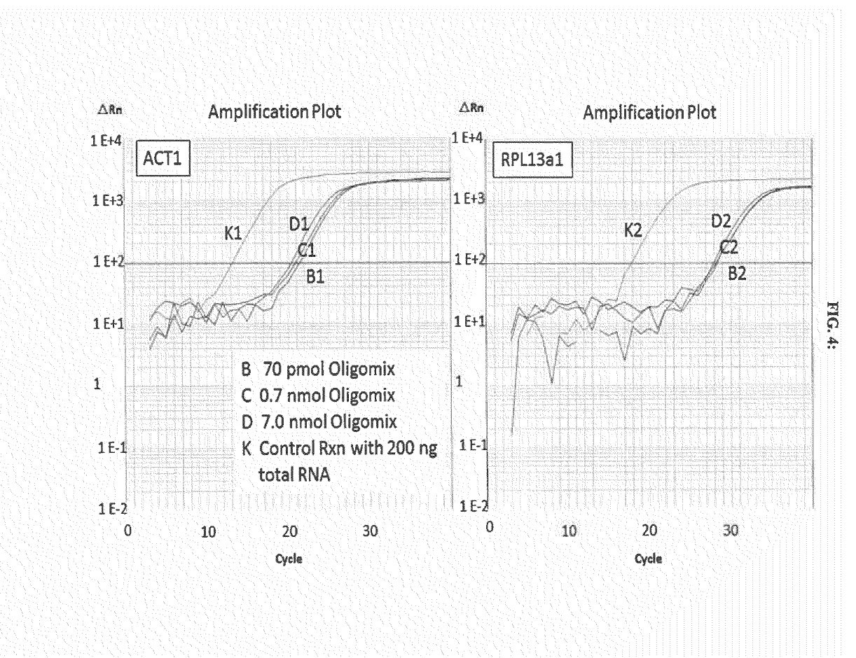

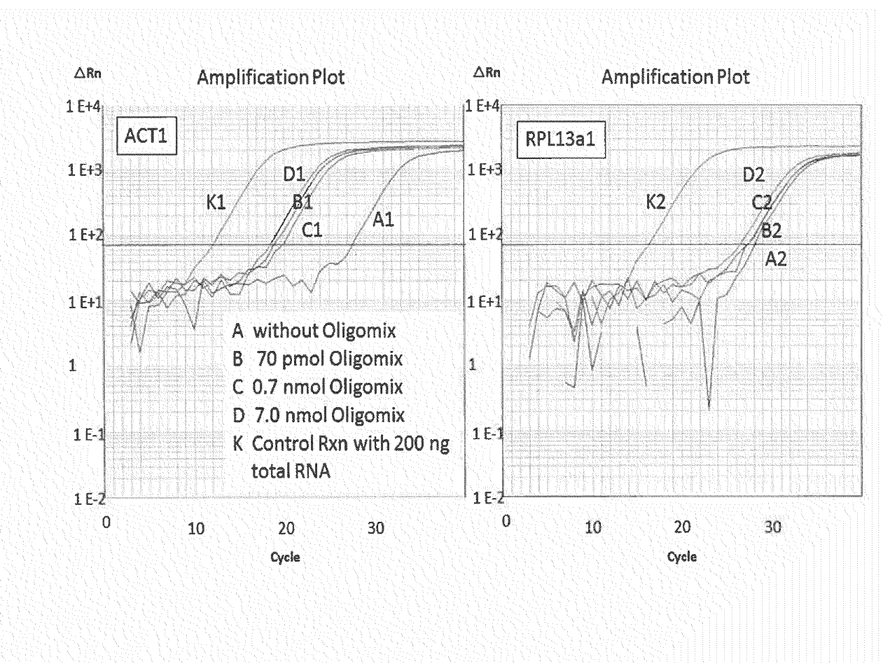

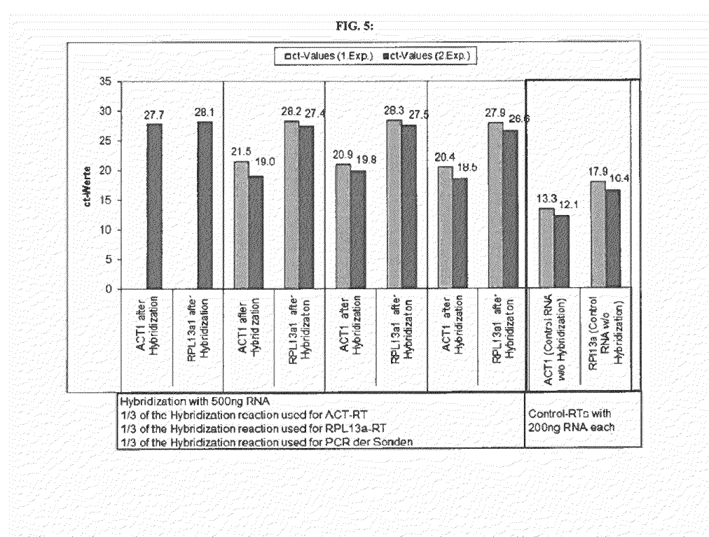

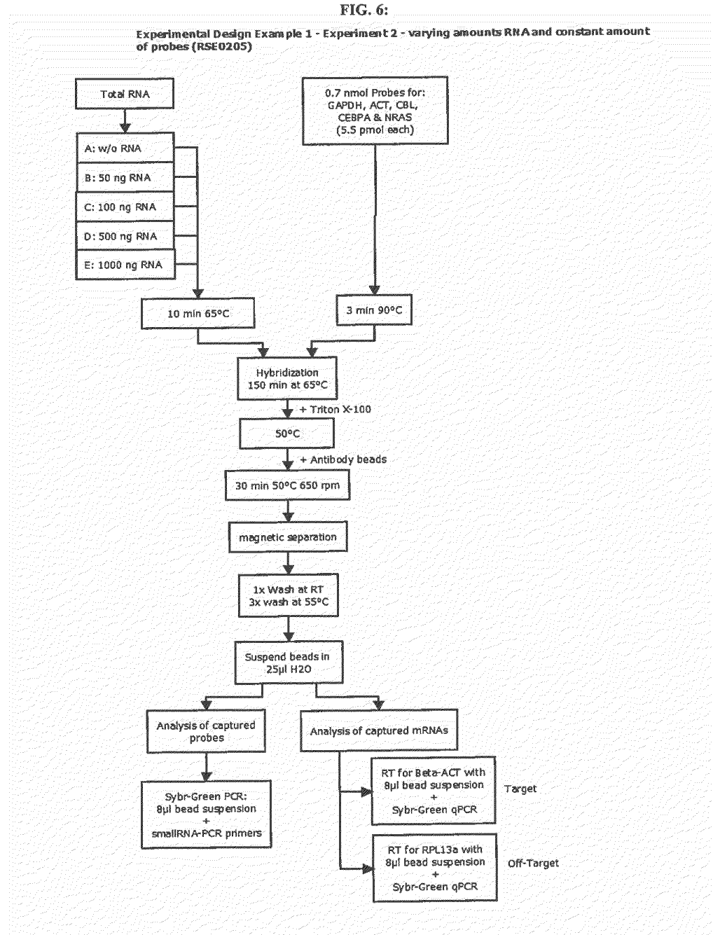

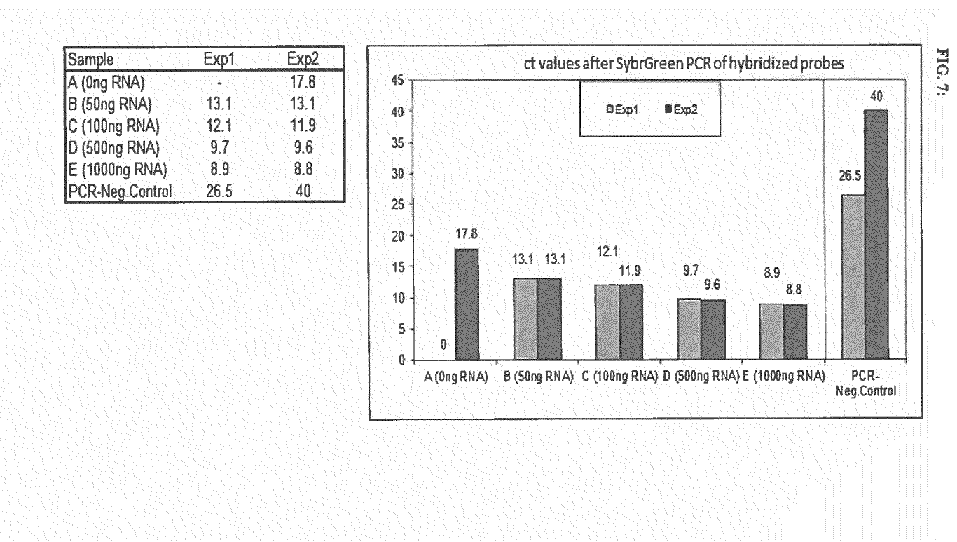

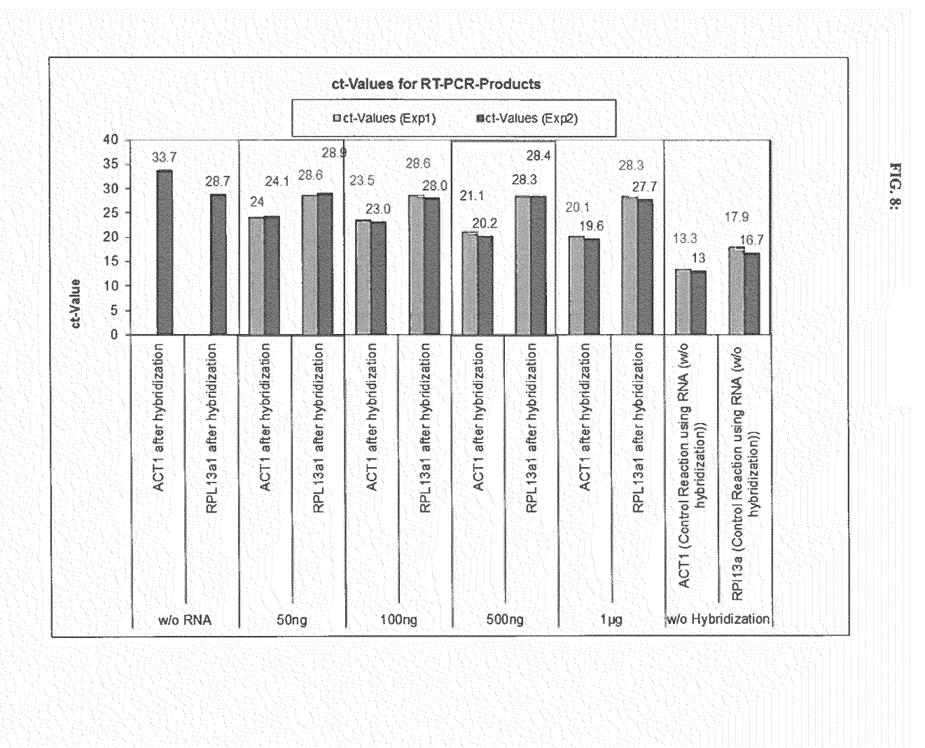

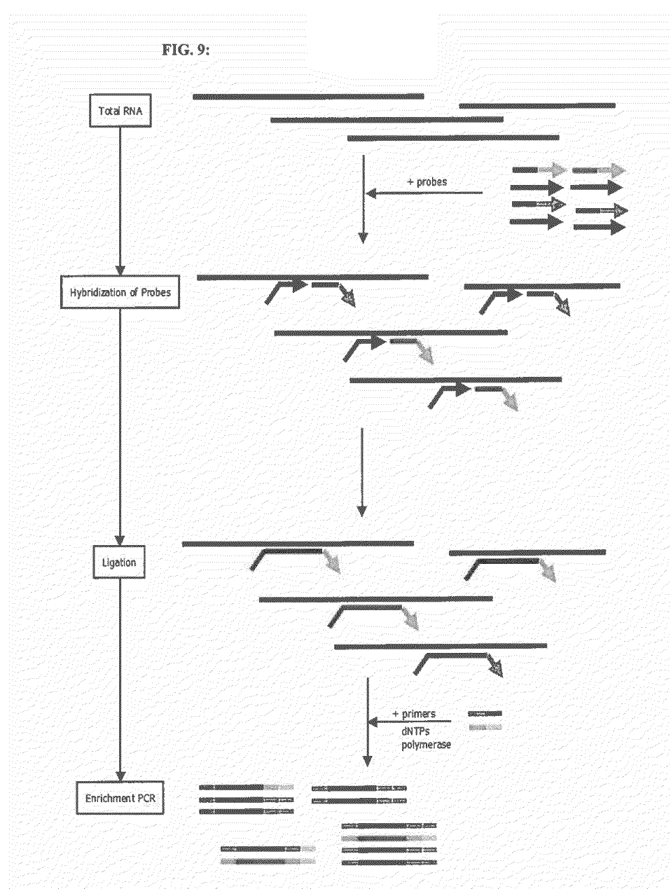

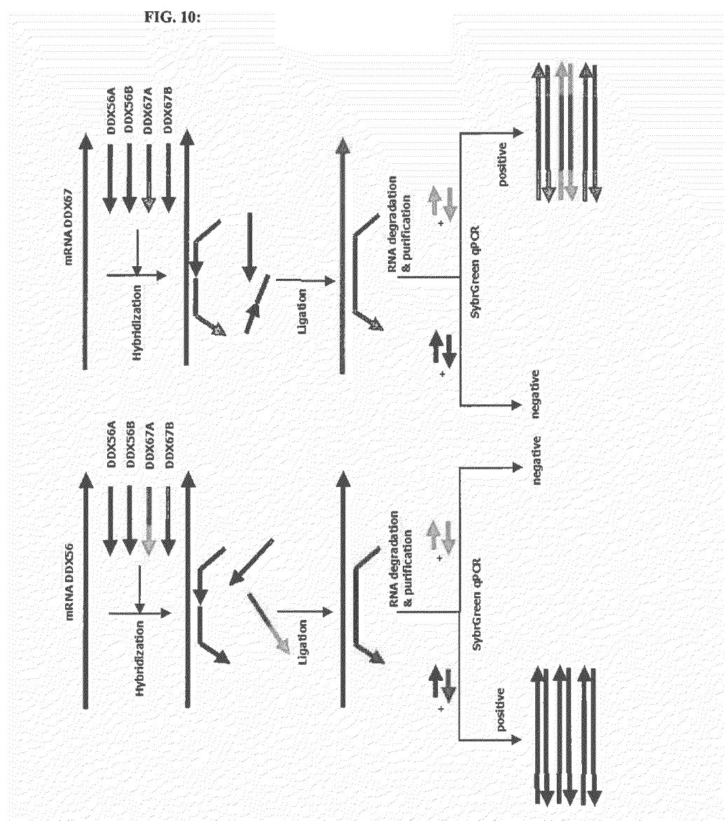

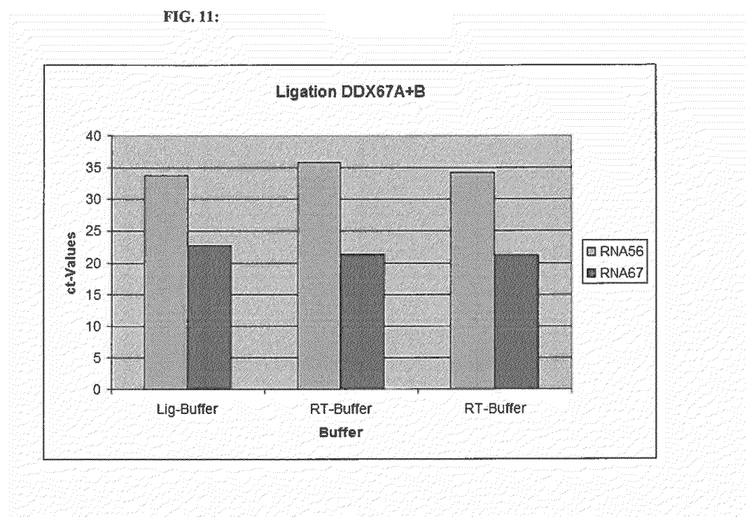

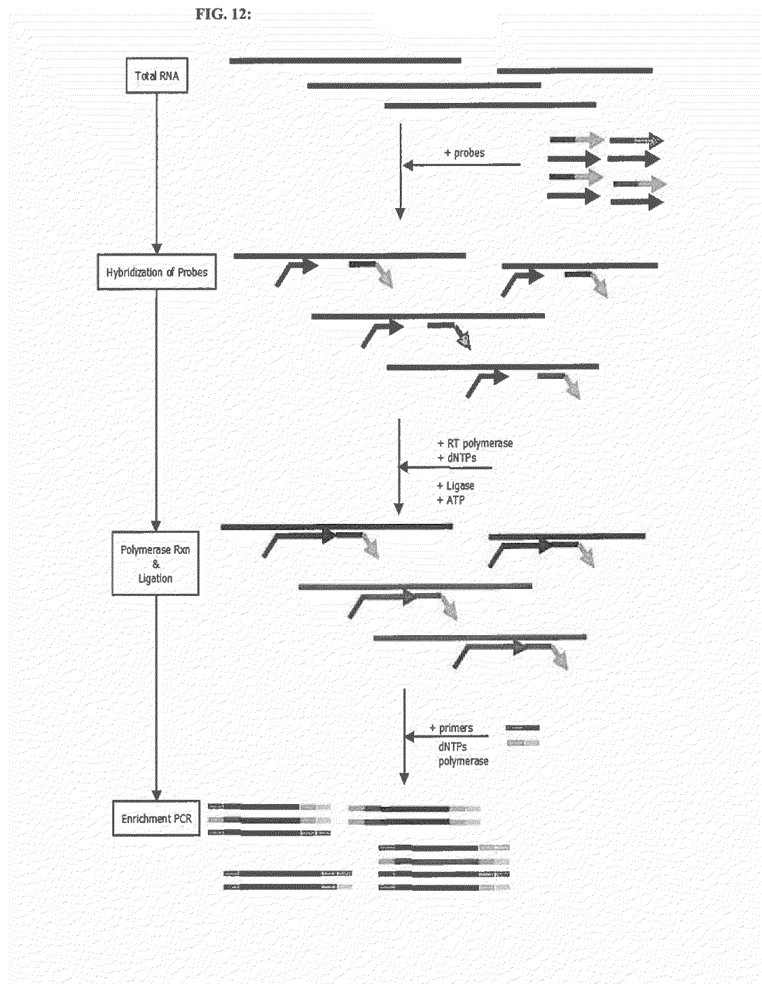

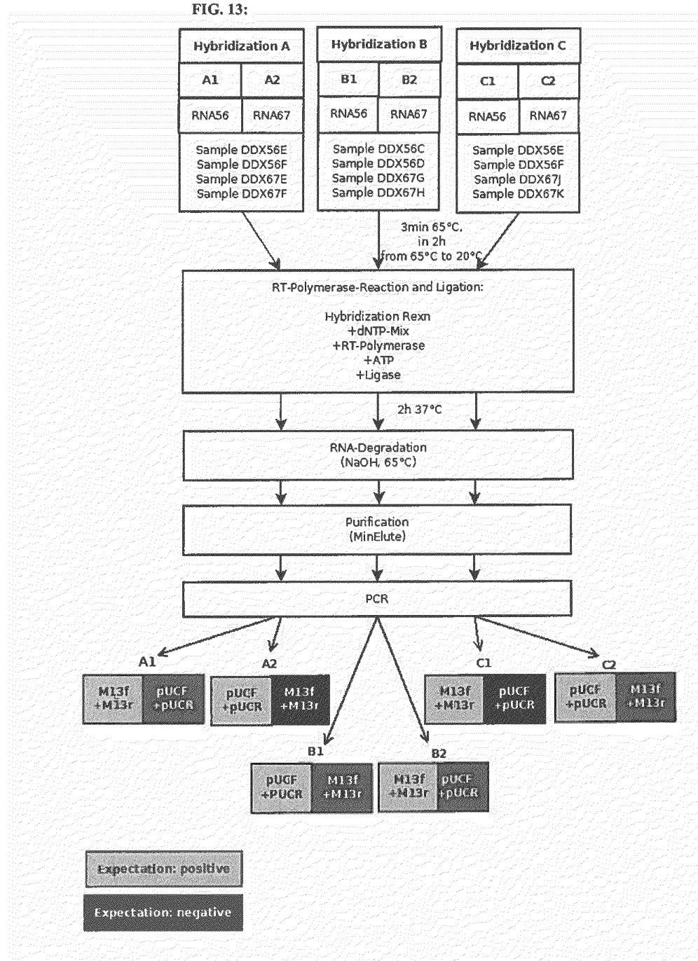

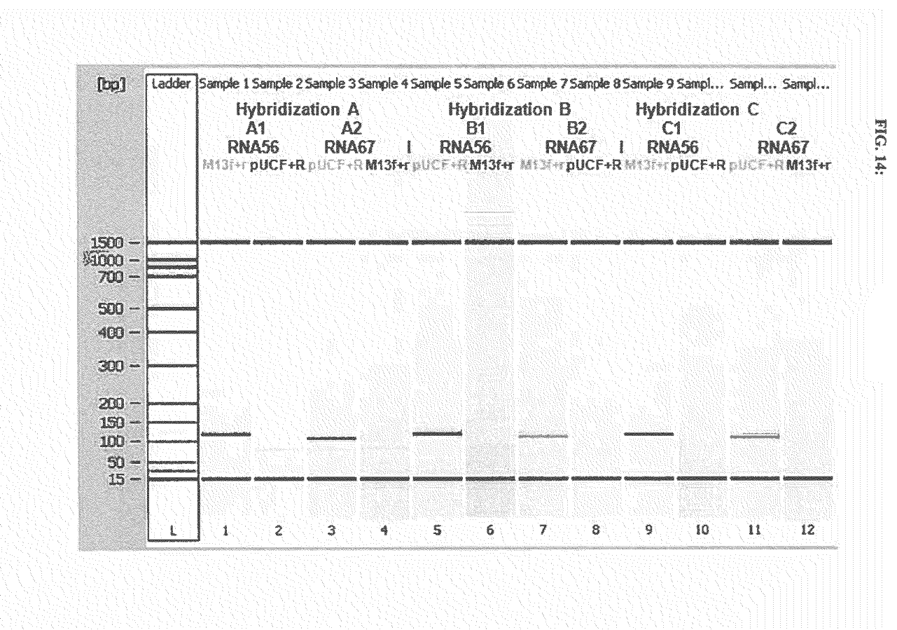

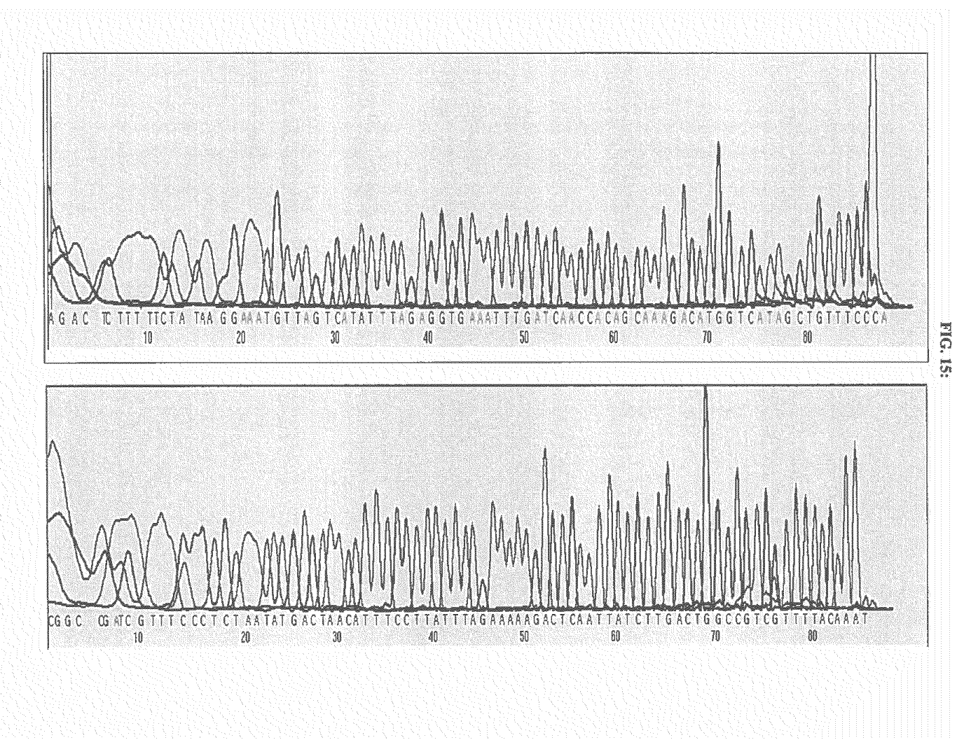

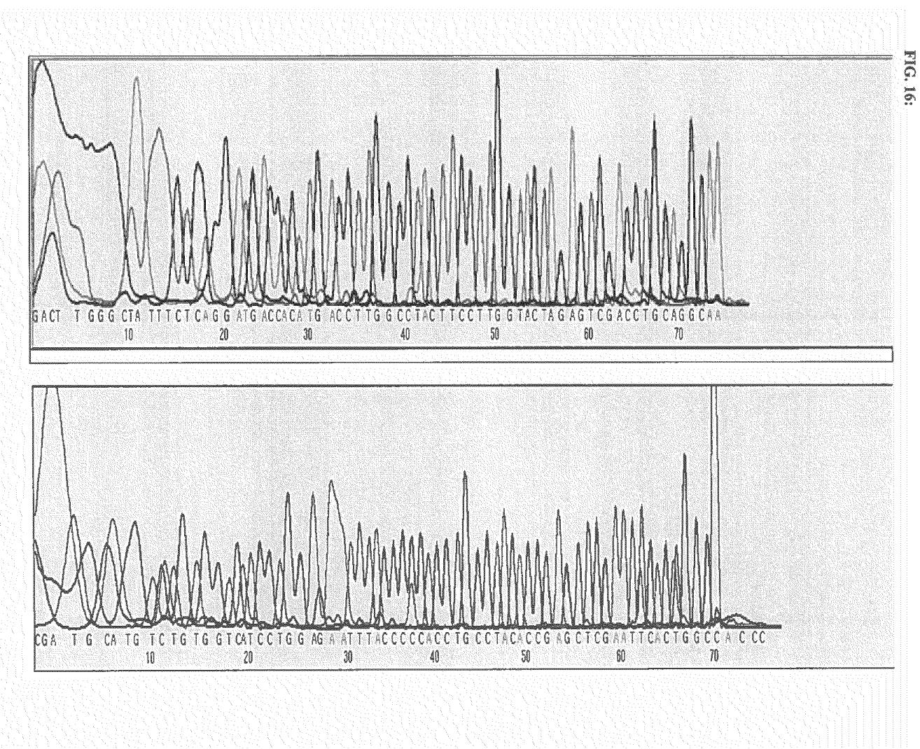

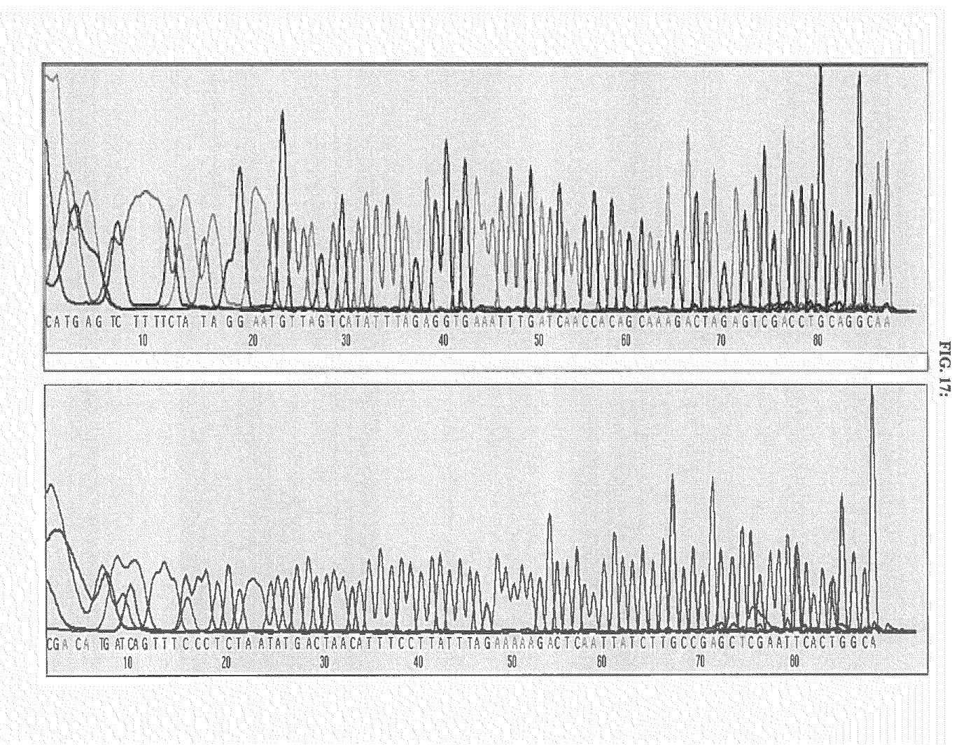

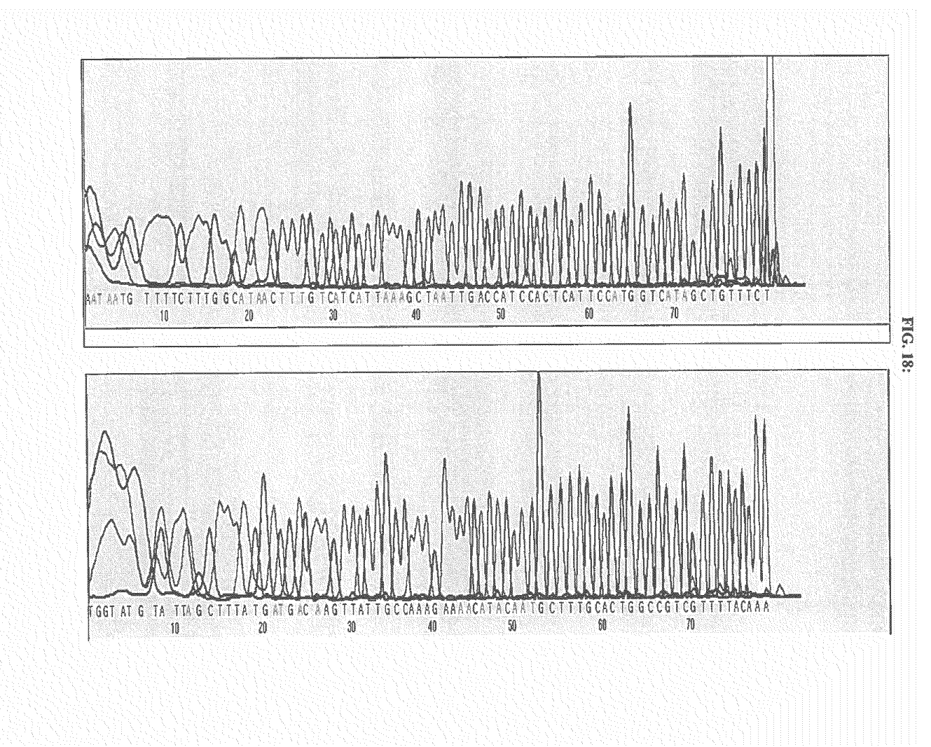

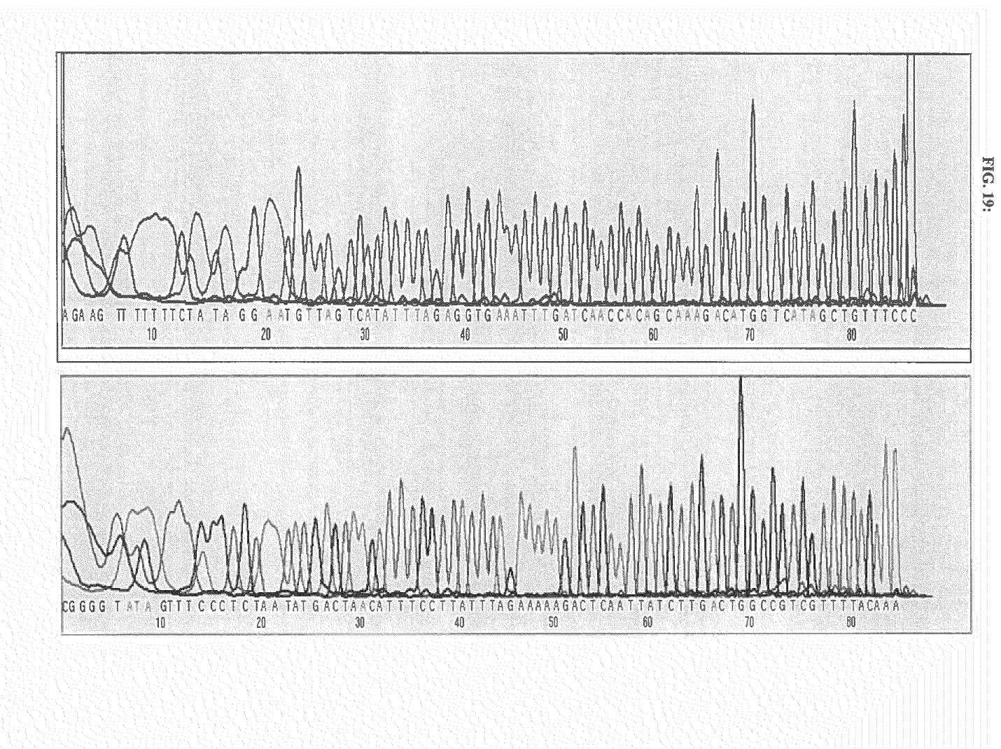

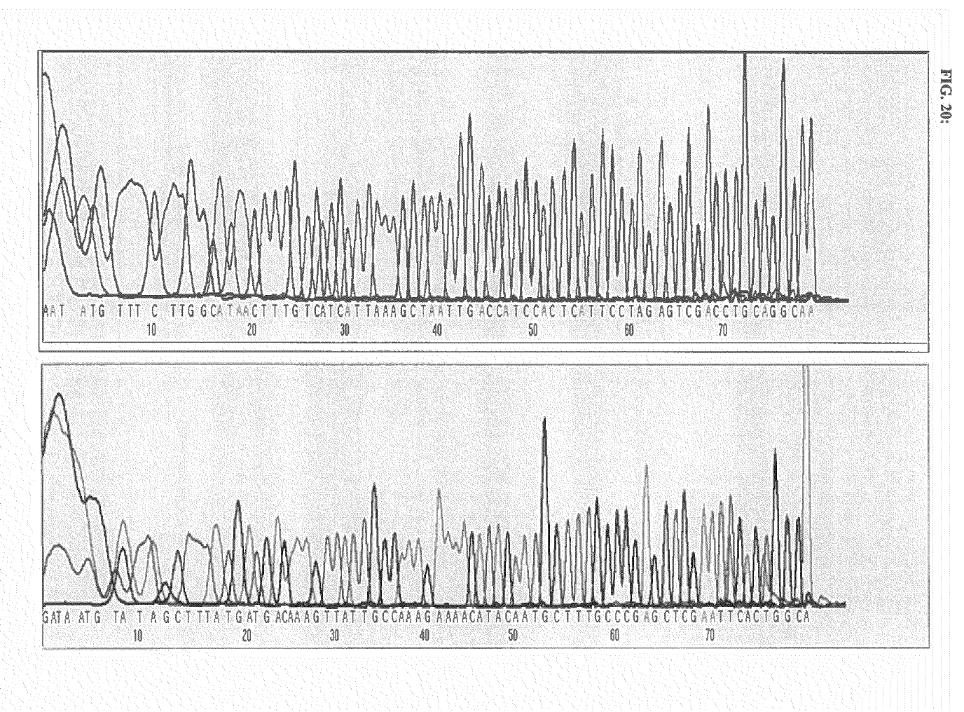

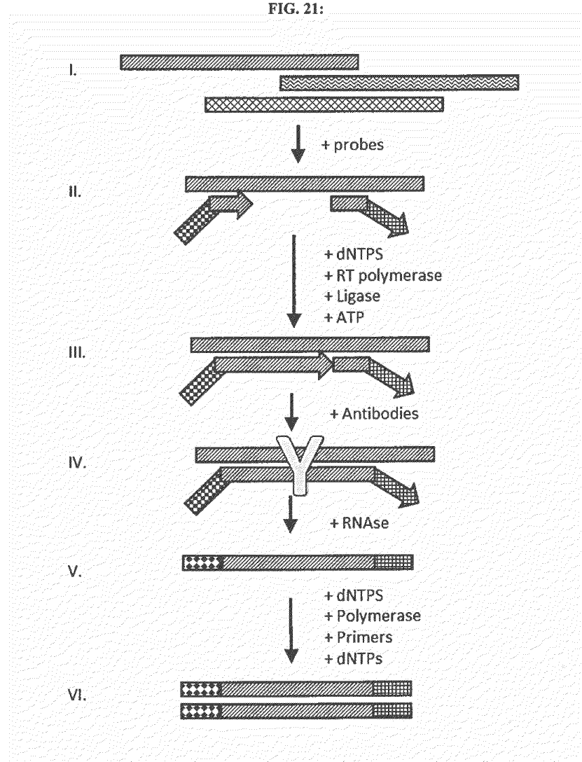

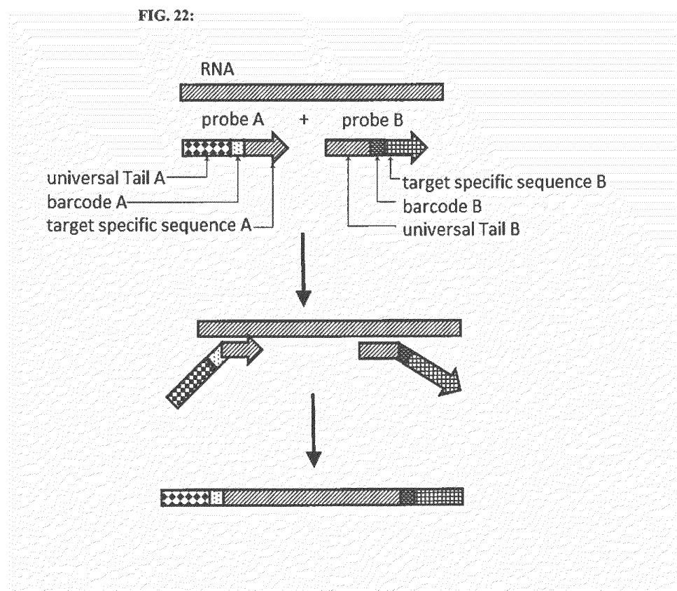

Specific DNA oligonucleotides containing universal adapters at the 5′ and 3′ ends were hybridized to targeted mRNAs and subsequently captured with antibodies, which bind DNA/RNA hybrids. After magnetic separation of the DNA/RNA hybrid molecules purified probe libraries were enriched by PCR prior to sequencing (FIG. 1).

DNA probes for hybridization with mRNAs of interest were designed specifically with comparable thermodynamic properties. Hybridization of the RNA with an excess of oligonucleotides followed by purification of the DNA/RNA hybrids allows quantification of the targeted mRNAs by determination of the number of DNA probes via sequencing.

By placing of probes on exon-exon junctions and adjustment of suitable hybridization conditions the selectivity to distinct mRNAs can be increased. Furthermore, it allows expression profiling of different splice variants of a mRNA.

### Example 1

Experiment 1:Hybridization of Total RNA with Varying Amounts of Hybrid Capture Probes

Sample: Total RNA from human T-cell Leukemia (Jurkat)

Target: mRNAs of following genes: GAPDH, ACTB, CBL, CEBPA1, NRAS

Off-Target: mRNAs of gene RPL13a

A description of the target and off-target sequences, probes for hybridization and primers for SybrGreen qPCR may be found in the appendix.

### Method and Results:

FIG. 2 gives an overview about the experimental workflow of example 1—experiment 1. Details of the experiment may be found in the appendix (RSE0204).

### Analysis of Captured Probes:

Although with 0.07nmol, 0.7 nmol and 7 nmol 2 magnitudes of different amounts of probes were hybridized with 500 ng total RNA in all cases comparable probe amounts were captured (FIG. 3). This indicates, that the amount of captured probes depends only on the amount of RNA in the hybridization reaction. Quantification of the captured oligo probes is suitable for determination of targeted mRNA expression levels.

Analysis of Captured mRNAs:

Similar ct values for captured target RNAs were obtained after hybridization with different amounts of probes indicate successful enrichment independent from the probes excess (FIG. 4). FIG. 5 summarizes the data in a column chart.

### Example 1

Experiment 2: Hybridization of varying amounts of total RNA with excess of hybrid capture probes was done as follow:

Sample: Total RNA from human T-cell Leukemia (Jurkat)

Target: mRNAs of following genes: GAPDH, ACTB, CBL, CEBPA1, NRAS

Off-Target: mRNAs of gene RPL13a

### Method and Results:

FIG. 6 summarizes the experiment workflow.

Analysis of Hybridized Probes by SybrGreen qPCR:

According to our expectation after hybridization of varying amounts of RNA with an excess of probe oligos the yields of both captured RNA and captured probes should correlate with the amounts of starting RNA. The yield of captured hybridization products increased with increasing RNA amounts, which is indicated by decreasing ct values after to SybrGreen-qPCR. In FIG. 7 the results of two independent experiments are summarized for the quantification of the captured probes. Doubling of the RNA amount resulted in a reduction of the ct value by around 1 for the captured probes.

### Example 2

mRNA Profiling by Ligation of Oligonucleotide Probes on RNA Templates:

### Principle

A probe consists of two tailed oligonucleotides. OligoB contains an universal 5′ tail and a target specific 3′ sequence. OligoA consists of an target specific 5′ end and an universal 3′ tail, Both tails are different in their base composition. In addition the 5′ end oligoA is phosphorylated. Both oligos match in direct neighborhood without a gap on their target RNA molecule allowing ligation of the 3′ end of oligoB with the phosphorylated 5′ end of oligoA. After hybridization and ligation fused oligo probes can be amplified via standard PCR using sequencer platform specific enrichment primers (FIG. 9). Probes which hybridize not in direct neighborhood and/or correct order will not be amplified by the enrichment PCR. Subsequently, enriched probes can be sequenced. Probe design follows classical primer design rules. Probe sequences should be specific for their target region. For example priming on different splice variations of the targeted mRNA is allowed whereas multiple priming on RNAs transcribed from different genes should be avoided. To prevent any unintended hybridization to genomic DNA, probably caused by insufficient RNA purification, both oligo probes A and B should hybridize with mRNA molecules on splice site junctions of two neighboring exons. This would enable expression profiling of different splice variants for the targeted genes by subsequent sequencing and clustering.

### Example 2

### Experiment 3: Ligation of Neighboring Oligonucleotides on RNA Templates

For evaluation if this idea is feasible, a model experiment was designed as following:

### Generation of Artificial RNAs:

Two PCR fragments with T7 RNA polymerase promoter sequence at one end (DDX56 and DDX67) were generated with tailed primers (LRT7_DDX06.p1_{—}01+LR_DDX5.q1_{—}01 and LR_DDX07.p1_{—}01+LRT7_DDX06.q1_{—}01, respectively) using human gDNA as template and subsequently transcribed in vitro using T7 RNA polymerase (see genomic DNA). Purified RNAs derived from both PCR fragments were used as template for hybridization and ligation experiments.

Tailed DNA probes consisting of 2 separate oligonucleotides, each were designed for their mRNA targets DDX56 and DDX67 as indicated in FIG. 9. Primer tails of probes for DDX56 and DDX67 differ in their sequence composition to enable detection of distinct ligation partners in a oligo mixture by amplification with probe specific PCR primers. A schematic workflow is shown in FIG. 10 and results after SybrGreen PCR are summarized for hybridization and ligation of the probe mixture on RNA template DDX67 in FIG. 11. The differences of ct values between ligated probe DDX67A+B and non-ligated probe DDX56A+B are comparable in different experiments after hybridization and ligation in ligation buffer containing 10 μM ATP. Similar results were obtained with RT polymerase buffer containing 10 μM ATP. Delta-cts between 10 to 15 indicate, that hybridization and ligation of DNA oligos on a RNA template was successful. Due to high PCR background after hybridization and ligation of oligo probe DDX56A+B experiments for demonstration of quantitative effects were not yet carried out. Also an improved experiment with increased selectivity was designed by combination of RNA/DNA hybridization with DNA synthesis and ligation of oligo probes on RNA templates (Example 3 experiments 4 and 5).

### Example 3

mRNA Profiling by Hybridization, Reverse Transcriptase Reaction and Subsequent Ligation of Tailed Oligonucleotide Probes on RNA Templates:

### Principle:

Probes are designed as in example 2, hut oligoA and B match in a distinct distance to their RNA target. Therefore, after hybridization a polymerase step is required to close the gap between both probe oligos prior to ligation (FIG. 12).

This additional DNA synthesis step offers some advantages in comparison to the previous examples:
The selectivity to generate probe fragments which can be amplified by PCR is improved. Generation of PCR amplifiable fragments requires correct priming of both probe oligos, correct filling of the gap between both oligos and ligation of the target specific align ends.
The number of probes for complete splicing profiling of targeted genes can be reduced, because one probe pair can be used to monitor different splicing events by location on conserved neighboring exon regions.
Furthermore, reduction of total number of oligos allows monitoring of more expression profiles in parallel.
Beside profiling of known splicing variants new splice forms can be discovered by sequencing.

### Example 3

### Experiment 4: Feasibility for one Step Polymerase and Ligase Reaction of Oligonucleotide Probes Hybridized to Targeted RNA

According to FIG. 12 for artificial RNAs DDX56 and DDX67 (see example 2) different probe oligos were designed which match on their RNA target in a distance of 33, 40 and 48 bases, respectively.

For RNA DDX56 probes DDX56C+D and DDX56E+F were synthesized with identical sequence homology to RNA DDX56, but different tails.

For RNA DDX67 3 probes were designed. Probes DDX67G+H and DDX67J+K differ only in the tail sequence, whereas probe DDX67E+F is located on a different position on RNA67. According to the probes and templates 3 different hybridization experiments A, B and C were set up (FIG. 13). After RT polymerase reaction and ligation fused probes were analyzed by capillary electrophoresis on Agilent 2100 (FIG. 14), SybrGreen qPCR (data not shown here; see appendix RSE0218 for results) and via Sanger sequencing (FIGS. 15 to 20).

### Example 3

Experiment 5: Model Experiment to Evaluate the Correlation between Targeted RNA Template Amount and Fused Oligo Probes

A mixture of probes DDX56E+F and DDX67J+K was hybridized to different amounts of RNA DDX56 and DDX67 (FIG. 16). Fusion products of the probes were quantified with SybrGreen qPCR (FIG. 17) and checked for their specificity by capillary electrophoresis.

Molecular barcodes are generated by introduction of random nucleotides between universal tail and target specific sequences of the probes allow differentiation between fragments derived from a target RNA molecule and copies generated during PCR amplification causing a sequence bias (FIG. 13). Such barcodes may be integrated in both probe A or B, respectively. For example: A barcode consisting of 5 random nucleotides allows differentiation of maximum 4⁵=1024 copies of a RNA molecules and two barcodes with 45 random nucleotides, each, allow differentiation of 4⁵*4⁵=1,048,576 molecules.

1. Method for determining the sequence and/or quantity of a ribonucleic acid in a composition, comprising the steps of:
i. providing a composition comprising one or more ribonucleic acids molecules (RNA).
ii. hybridizing to said one or more RNAs, one or more two-part nucleic acid hybridization probes, wherein each probe comprises,
a. a first nucleic acid molecule (DNA) with a 3′-tail, wherein said tail does not hybridize to an RNA in the composition,
b. a second nucleic acid molecule (DNA) with a 5′-tail, wherein said tail does not hybridize to an RNA in the composition,
c. wherein said first and said second nucleic acid molecules when and if hybridized to their target RNA lie on one single stranded RNA molecule separated from each other by between 2 and 1000 nucleotides,
iii. covalently linking the hybridized first nucleic acid molecule to the hybridized second nucleic acid, wherein the linking is done by means of reverse transcription and subsequent ligation,
iv. amplifying the linked molecules with primers that are specific for said first 3′-tail of said first nucleic acid molecule and said second 5′-tail of said second nucleic acid molecule,
v. sequencing the amplification products, wherein prior to the amplification step (iv)
vi. the hybrids of target RNA and linked molecules of step (iii) are isolated by capturing the hybrids with an antibody that is specific for a DNA/RNA hybrid.

2. Method according to claim 1, wherein the RNA is enzymatically digested prior to the amplification step (iv).

3. Method according to claim 1, wherein the tail of the first and second nucleic acid molecules is between 10 and 50 nucleotides.

4. Method according to claim 1, wherein the first nucleic acid molecule and/or second nucleic acid molecule comprise a further barcode sequence, determined not to bind the target RNA and wherein said sequence is between 3 and 20 in length.

5. Method according to claim 1, wherein the first nucleic acid molecule and the second nucleic acid molecule are specific for a nucleic acid sequence selected from the group of a human nucleic acids sequence, a viral sequence, a bacterial sequence, an animal sequence and a plant sequence.

6. Method according to claim 5, wherein the sequence is an mRNA sequence, an exon-exon junction and/or a 5′ and 3′ UTR region.

7. Method according to claim 1, wherein the next generation sequencing method applied is selected from the group of sequencing by synthesis, pyrosequencing, Sanger sequencing, sequencing by oligo ligation, semiconductor technology or single molecule real-time (SMRT™) sequencing.

8. Method according to claim 1, wherein the concentration of the first nucleic acid molecule with a 3′-tail wherein said tail does not hybridize to an RNA in the composition and the second nucleic acid molecule with a tail wherein said tail does not hybridize to an RNA in the composition is between 1 fM and 1000 nM.

9. Kit comprising
i. a first nucleic acid molecule (DNA) with a 3′-tail wherein said tail does not hybridize to an RNA in the composition,
ii. a second nucleic acid molecule (DNA) with a 5′-tail wherein said tail does not hybridize to an RNA in the composition, wherein said first and said second nucleic acid molecules when and if hybridized to their target RNA lie on one single stranded RNA molecule separated from each other by between 2 and 1000 nucleotides,
iii. an antibody which is specific for an RNA/DNA duplex hybrid molecule, and
iv. a reverse transcriptase and/or a DNA ligase


SEQUENCE LISTING (10 sequences)

SEQ ID NO: 1 - DNA, 88 bp, Artificial Sequence
  Hybridization Probe
agactctttttctataaggaaatgttagtcatatttagaggtgaaatttgatcaaccacagcaaagacatggtcatagctgtttccca

SEQ ID NO: 2 - DNA, 86 bp, Artificial Sequence
  Hybridization Probe
cggccgatcgtttccctctaatatgactaacatttccttatttagaaaaagactcaattatcttgactggccgtcgttttacaaat

SEQ ID NO: 3 - DNA, 74 bp, Artificial Sequence
  Hybridization Probe
gacttgggctatttctcaggatgaccacatgaccttggcctacttccttggtactagagtcgacctgcaggcaa

SEQ ID NO: 4 - DNA, 74 bp, Artificial Sequence
  Hybridization Probe
cgatgcatgtctgtggtcatcctggagaatttacccccacctgcctacaccgagctcgaattcactggccaccc

SEQ ID NO: 5 - DNA, 87 bp, Artificial Sequence
  Hybridization Probe
catgagtcttttctataggaatgttagtcatatttagaggtgaaatttgatcaaccacagcaaagactagagtcgacctgcaggcaa

SEQ ID NO: 6 - DNA, 88 bp, Artificial Sequence
  Hybridization Probe
cgacatgatcagtttccctctaatatgactaacatttccttatttagaaaaagactcaattatcttgccgagctcgaattcactggca

SEQ ID NO: 7 - DNA, 80 bp, Artificial Sequence
  Hybridization Probe
aataatgttttctttggcataactttgtcatcattaaagctaattgaccatccactcattccatggtcatagctgtttct

SEQ ID NO: 8 - DNA, 79 bp, Artificial Sequence
  Hybridization Probe
tggtatgtattagctttatgatgacaagttattgccaaagaaaacatacaatgctttgcactggccgtcgttttacaaa

SEQ ID NO: 9 - DNA, 79 bp, Artificial Sequence
  Hybridization Probe
aatatgtttcttggcataactttgtcatcattaaagctaattgaccatccactcattcctagagtcgacctgcaggcaa

SEQ ID NO: 10 - DNA, 80 bp, Artificial Sequence
  Hybridization Probe
gataatgtatagctttatgatgacaaagttattgccaaagaaaacatacaatgctttgcccgagctcgaattcactggca